(19) United States
(12) Patent Application Publication
(10) Pub. No.: US 2012/0035683 A1
(43) Pub. Date: Feb. 9, 2012

(54) METHOD FOR TREATING DEGENERATIVE DISC DISEASE USING NONINVASIVE CAPACITIVELY COUPLED ELECTRICAL STIMULATION DEVICE

(76) Inventors: Bruce J. Simon, Mountain Lakes, NJ (US); Jean C. Gan, Morris Township, NJ (US)

(21) Appl. No.: 13/081,443

(22) Filed: Apr. 6, 2011

Related U.S. Application Data
(63) Continuation-in-part of application No. 11/176,588, filed on Jul. 7, 2005, now Pat. No. 8,082,038.
(60) Provisional application No. 60/586,745, filed on Jul. 9, 2004.

(51) Int. Cl. A61N 1/36

(52) U.S. Cl. 607/50

(57) ABSTRACT

A method for treatment of degenerative disc disease using capacitively coupled electrical stimulation. In one embodiment, a subject diagnosed as having degenerative disc disease is treated by placing first and second electrodes on the subject's body at the site of an identified disc in a state of degenerative disc disease, and applying an electric field to the identified disc via the first and second electrodes with the intent to treat the degenerative disc disease. The electric field is created with an electrical signal having a frequency within a range of 20 to 100 kHz and having a symmetrical waveform with an amplitude within a range of 0.1 to 20 volts peak-to-peak, preferably a frequency of approximately 60 kHz and an amplitude of approximately 5 volts peak-to-peak.

### BACKGROUND OF THE INVENTION

This invention relates to methods for treating degenerative disc disease, and more particularly to methods for treating degenerative disc disease noninvasively.

Degenerative disc disease (DDD), an irreversible process, is the most common cause of back pain. The intervertebral disc consists of a gelatinous nucleus pulposus encapsulated by a fibrous annulus fibrosus and the end-plates. The nucleus pulposus plays an important role in weight transmission. When this gel-like fluid is subjected to load, the nucleus pulposus is pressurized deforming to establish an equilibrating pressure between the annulus fibrosus and vertebral end-plates. However, with maturation, the tissue loses its gel-like properties becoming less hydrated and ultimately more fibrous. It is thus less able to transmit the load by exerting pressure on the annulus fibrosus. As a result, the annulus fibrosus undergoes greater deformation, and a greater share of the vertical load is borne directly by the annulus fibrosus. These changes result in the development of cracks and cavities within the annulus fibrosus leading to degeneration.

There are 3 therapeutic approaches to date:

1) Anti-neuralgic and anti-inflammatory;

2) Minimally invasive percutaneous treatments such as discectomy, intradiscal electrothermy, nucleoplasty and percutaneous radiofrequency application; and

3) Major surgeries such as fusions, laminectomy and nucleus/disc replacements.

Anti-neuralgic and anti-inflammatory methods treat the symptoms but do not eliminate the problem. Minimally invasive percutaneous treatments such as those identified above temporarily improve the symptoms, but they do not prevent the progression of DDD and may even accelerate it in the long term. Major surgeries such as those identified above eliminate structures, risk nerve injury during the operation, also risk morbidity, and may result in accelerated disc degeneration at adjacent segments in the long term.

### SUMMARY OF THE INVENTION

The present invention provides for treatment of degenerative disc disease as a new indication for capacitively coupled electrical stimulation. According to one aspect of the present invention, a subject diagnosed as having degenerative disc disease is treated by placing first and second electrodes on the subject's body at the site of an identified disc in a state of degenerative disc disease, and applying an electric field to the identified disc via the first and second electrodes with the intent to treat the degenerative disc disease. The electric field is created with an electrical signal having a frequency within a range of 20 to 100 kHz and having a symmetrical waveform with an amplitude within a range of 0.1 to 20 volts peak-to-peak.

Treatment in accordance with the invention regenerates the intervertebral disc or prevents further degeneration of the disc and thus restores the mechanical properties of the spine or reduces pain resulting from disc degeneration (e.g., discogenic pain). The device may be used as an adjunct to other treatments such as implantation of cells (e.g., stem cells from various sources and intervertebral disc cells), biological factors (e.g., growth factors, peptides), scaffolds or materials (e.g., collagen, polymers, ceramics) and gene therapy.

Among other advantages of the present invention, it is a noninvasive and simple approach to relieving pain associated with the disc. It is simple to use, surgery is not required, the treatment can be administered early to prevent further progression of DDD, and it may regenerate the disc. Regeneration of the disc restores the mechanical properties of the spine, and thus, unlike other treatments such as spinal fusions, the present invention will not cause accelerated disc degeneration at adjacent segments in the long term.

These and other objects and advantages of the present invention will be more apparent upon reading the following detailed description in conjunction with the accompanying drawings.

### BRIEF DESCRIPTION OF THE DRAWINGS

FIG. 1 is a rear view of the lower torso of a human subject with a pair of electrodes disposed on the skin proximate to the spine for treatment of degenerative disc disease according to one embodiment of the present invention.

FIG. 2 is a cross-sectional view of the portion of the spine proximate to the skin electrodes of FIG. 1.

### DESCRIPTION OF PREFERRED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device and such further applications of the principles of the invention as illustrated therein being contemplated as would normally occur to one skilled in the art to which the invention relates.

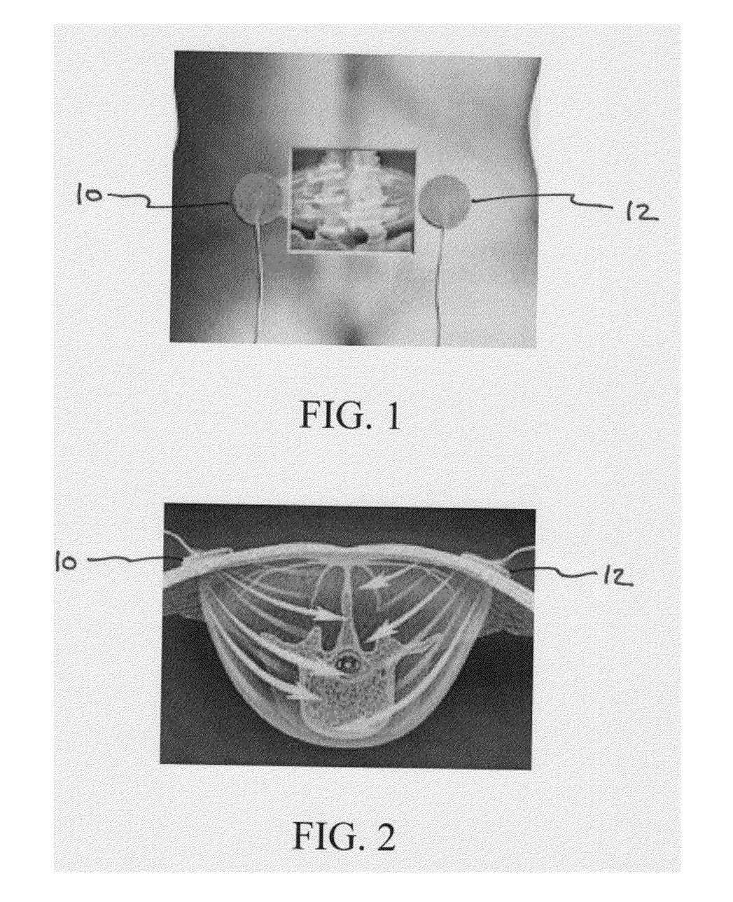

The present invention provides a new method of treating degenerative disc disease (DDD) by using a noninvasive capacitively coupled electrical stimulation device that is compact, light and easy to use, and yet highly effective in operation. In the method according to one aspect of the present invention, two or more electrodes, e.g., electrodes 10 and 12 in FIGS. 1 and 2, are applied noninvasively on the skin at the site of pain or location of the intervertebral disc to be treated. For example, an electrode may be placed on either side of the spine at the level of a disc to be treated, with spacing of 4″ to 6″ between the electrodes. Suitable electrodes include those used with the SpinalPak spinal fusion stimulator, commercially available from EBI, L.P. As those skilled in the art will understand from the foregoing, the capacitively coupled electrical stimulation is provided to an intervertebral disc that has not been treated by spinal fusion, which is a known surgical procedure in which the vertebrae adjacent to a degenerated disc are fused together so as to eliminate the pain caused by the DDD by immobilizing the vertebrae themselves. In the present method, a SpinalPak may be used to provide the capacitively coupled electrical stimulation, but the electrical stimulation is applied as an alternative, not an adjunct, to spinal fusion. That is, it is applied instead of, not in addition to, spinal fusion surgery.

The capacitively coupled electrical stimulation may be applied as the sole corrective therapy for DDD, i.e., without any surgery, such as the other major surgeries identified above, or other treatments, or it may be applied in addition to non-fusion surgical procedures and minimally invasive procedures in which disc tissue is retained, and/or in addition to medication or the other treatments identified above.

The electrodes are connected to a signal generator which emits a signal with a frequency of 20 to 100 kHz and having a symmetrical waveform with an amplitude of 0.1 to 20 volts peak-to-peak. Examples of signals are described in patent application Ser. No. 10/041,850, entitled Non-Invasive Capacitively Coupled Electrical Stimulation Device For Treatment Of Soft Tissue Wounds, filed Jan. 7, 2002 in the name of Jean C. Gan et al., which application is assigned to the assignee of the present invention and incorporated herein by reference. One preferred form of the signal is a symmetrical sine wave with a frequency of approximately 60 kHz and an amplitude of approximately 5 volts peak-to-peak. A bipolar DC signal with a symmetrical step or a triangular waveform may be useful in certain applications of the present invention. The signal is preferably applied intermittently but may be applied continuously for a period of time.

While the invention is not limited to any one theory of operation, it is believed that the invention promotes disc tissue regeneration and/or inhibits further progression of DDD by enhancing the proliferation and matrix synthesis of chondrocytes and stimulating the production of inflammatory cytokine antagonists, such as TGF-β. As indicated above, the invention may be used as an adjunct to other treatments such as implantation of cells, biological factors, scaffolds or materials, and gene therapy. The following additional patents and publications are hereby incorporated by reference along with all references cited therein: U.S. Pat. Nos. 4,467,808 and 4,535,775 to Brighton et al.; International Application No. PCT/US01/05991 in the name of Brighton et al.; Brighton Carl T., et al., “Treatment of Castration-Induced Osteoporosis by a Capacitively Coupled Electrical Signal in Rat Vertebrae,” J. Bone and Joint Surg., 71-A: 228-236, 1989; Brighton, Carl T., et al., “Treatment of Denervation/Disuse Osteoporosis in the Rat with a Capacitively Coupled Electrical Signal: Effects on Bone Formation and Bone Resorption,” J. Orthop. Res., 6:676-684, 1988.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

We claim:

1. A nonsurgical method of treating degenerative disc disease, comprising:
identifying an unfused disc in a state of degenerative disc disease in a subject; and
nonsurgically treating the degenerative disc disease by applying capacitively coupled electrical stimulation to the identified unfused disc, intending to promote generation of disc tissue as an alternative to spinal fusion, the electrical stimulation characterized by an electric field created with an electrical signal having a frequency within a range of 20 to 100 kHz and having a symmetrical waveform with an amplitude within a range of 0.1 to 20 volts peak-to-peak.

2. The method of claim 1, wherein said electrical signal has a frequency of approximately 60 kHz and an amplitude of approximately 5 volts peak-to-peak.

3. The method of claim 2, wherein said electrical signal is a sine wave.

4. The method of claim 2, wherein said electrical signal is a bipolar DC signal with a square waveform.

5. The method of claim 2, wherein said electrical signal is a bipolar DC signal with a triangular waveform.

6. A nonsurgical method of treating degenerative disc disease, comprising:
placing first and second electrodes on the body of a subject at the site of a disc in a state of degenerative disc disease; and
applying an electric field to said disc noninvasively via said first and second electrodes as an alternative, not an adjunct, to spinal fusion.

7. The method of claim 6, wherein said electric field is created by supplying a voltage across said electrodes at a frequency within a range of 20 to 100 kHz and an amplitude within a range of 0.1 to 20 volts peak-to-peak.

8. The method of claim 7, wherein said frequency is approximately 60 kHz and said amplitude is approximately 5 volts peak-to-peak.

9. The method of claim 8, wherein said voltage is sinusoidal.

10. The method of claim 8, wherein said voltage is a square wave.

11. The method of claim 8, wherein said voltage is a triangular wave.

12. A nonsurgical method of treating degenerative disc disease, comprising applying capacitively coupled electrical stimulation to a human intervertebral disc in a state of degenerative disc disease untreated by spinal fusion surgery.

13. The method of claim 12, wherein said electrical stimulation is characterized by an electric field created with an electrical signal which has a frequency of approximately 60 kHz and an amplitude of approximately 5 volts peak-to-peak.

14. The method of claim 13, wherein said electrical signal is a sine wave.

15. The method of claim 13, wherein said electrical signal is a bipolar DC signal with a square waveform.

16. The method of claim 13, wherein said electrical signal is a bipolar DC signal with a triangular waveform.

17. A nonsurgical method of treating degenerative disc disease, comprising applying capacitively coupled electrical stimulation to a human intervertebral disc in a state of degenerative disc disease between two adjacent vertebrae which are capable of substantially normal relative motion.

18. A nonsurgical method of treating degenerative disc disease, comprising applying capacitively coupled electrical stimulation to a human intervertebral disc in a state of degenerative disc disease, wherein said disc is in a structurally native state between two adjacent vertebrae which are unconstrained by orthopedic hardware.
